(19) United States
(12) Patent Application Publication
(10) Pub. No.: US 2011/0064674 A1
(43) Pub. Date: Mar. 17, 2011

(54) LUMINESCENT MULTIMODAL NANOPARTICLE PROBE SYSTEM AND METHOD OF MANUFACTURE THEREOF

(75) Inventors: Marcel Bruchez, Pittsburgh, PA (US); Traian Sarbu, Pittsburgh, PA (US)

(73) Assignee: CARNEGIE MELLON UNIVERSITY, Pittsburgh, PA (US)

(21) Appl. No.: 12/994,150

(22) PCT Filed: May 21, 2009
(86) PCT No.: PCT/US09/44917

§ 371 Date: Nov. 22, 2010

Related U.S. Application Data
(60) Provisional application No. 61/128,332, filed on May 21, 2008.

(51) Int. Cl. A61K 49/18; B05D 3/00; B05D 1/36; C12Q 1/00; B32B 1/00; A61P 43/00

(52) U.S. Cl. 424/9.3; 427/2.11; 427/331; 427/402; 435/4; 428/403

(57) ABSTRACT

A method of manufacture of a luminescent nanoparticle probe system includes forming a bismuth sulfide core; and coating the bismuth sulfide core with a shell.

### CROSS-REFERENCE TO RELATED APPLICATION(S)

This application claims the benefit of U.S. Provisional Patent Application Ser. No. 61/128,332 filed May 21, 2008, and the subject matter thereof is incorporated herein by reference thereto.

### GOVERNMENT RIGHTS

This invention was made with Government support under grant No. NiH-R01-EB000364 awarded by the National Institutes of Health. The government has certain rights in the invention.

### TECHNICAL FIELD

The present invention relates generally to a nanoparticle probe, and more particularly to a luminescent multi-modality nanoparticle probe system.

### BACKGROUND ART

Luminescent nanoparticles show tremendous promise for in vitro and in vivo biochemical detection and imaging, such as imaging of biochemical markers for cancer and infectious diseases. Their unique optical properties have prompted a rapid expansion of their applications, especially in living animals, with potential utility for clinical use. However, most luminescent nanoparticles contain toxic elements such as cadmium, arsenic, indium and mercury, thus raising growing concerns regarding environmental and safety issues. Detailed safety data are largely unavailable for these particles, thus restricting their use to preclinical/non-human applications.

One important requirement for nanoparticles for use in living organisms is efficient luminescent emission in the near infrared range (NIR), in the optical window between hemoglobin absorption and water absorption. Further, operation at long NIR wavelengths reduces Rayleigh scattering in tissues. Operation in the NIR thus increases the tissue depth for imaging and reduces autofluorescence background from native proteins such as collagens, porphyrins, and flavins.

Additionally there is a need for multi-modality nanoparticles: particles that exhibit properties that enable detection by different means of detection such as optical, x-ray, and nuclear magnetic resonance detection.

Thus, a need still remains for non-toxic multi-modality nanoparticles for biochemical detection in vitro and in vivo. In view of the need for quantitative biochemical detection in biochemical specimens and living organisms, it is increasingly critical that answers be found to these problems. In view of the ever-increasing commercial competitive pressures, along with growing consumer expectations and the diminishing opportunities for meaningful product differentiation in the marketplace, it is critical that answers be found for these problems. Additionally, the need to reduce costs, improve efficiencies and performance, and meet competitive pressures adds an even greater urgency to the critical necessity for finding answers to these problems.

Solutions to these problems have been long sought but prior developments have not taught or suggested any solutions and, thus, solutions to these problems have long eluded those skilled in the art.

### DISCLOSURE OF THE INVENTION

The present invention provides a method of manufacture of a luminescent nanoparticle probe system including forming a bismuth sulfide core; and coating the bismuth sulfide core with a shell.

In addition, the present invention provides a luminescent nanoparticle probe system, including: a bismuth sulfide core; and a shell coating the bismuth sulfide core.

Certain embodiments of the invention have other steps or elements in addition to or in place of those mentioned above. The steps or element will become apparent to those skilled in the art from a reading of the following detailed description when taken with reference to the accompanying drawings.

### BRIEF DESCRIPTION OF THE DRAWINGS

FIG. 1 is a plot of luminescence emission wavelength for a luminescent nanoparticle probe system 102 in one embodiment of the present invention compared to the emission of nanoparticles of different composition.

FIG. 2 is a synthesis scheme for manufacturing the luminescent nanoparticle probe system in one embodiment of the present invention.

FIG. 3 is a high-resolution Transmission Electron Microscope (TEM) image showing the bismuth sulfide core in the luminescent nanoparticle probe system of the present invention.

FIG. 4 is a size distribution plot of the bismuth sulfide core.

FIG. 5 is X-ray diffraction data for the bismuth sulfide core of the luminescent nanoparticle probe system of the present invention.

FIG. 6 is fluorescence emission spectra of the bismuth sulfide core in the luminescent nanoparticle probe system of the present invention.

FIG. 7 is X-ray (CT) contrast data for the bismuth sulfide core compared to that of a cadmium telluride nanoparticle.

FIG. 8 is an image showing fluorescence of the bismuth sulfide core in the luminescent nanoparticle probe system.

FIG. 9 is an image of a nude mouse that has been administered intradermally a saline solution containing the bismuth sulfide core (i.e., a suspension of these nanoparticles).

FIG. 10 is a schematic diagram of the luminescent nanoparticle probe system in one embodiment of the present invention.

FIG. 11 is a luminescent nanoparticle probe system incorporating the luminescent nanoparticle probe system of FIG. 10 and an external molecule.

FIG. 12 a flow chart of a method of manufacture of a luminescent nanoparticle probe system in a further embodiment of the present invention.

### BEST MODE FOR CARRYING OUT THE INVENTION

The following embodiments are described in sufficient detail to enable those skilled in the art to make and use the invention. It is to be understood that other embodiments would be evident based on the present disclosure, and that system, process, or mechanical changes may be made without departing from the scope of the present invention.

In the following description, numerous specific details are given to provide a thorough understanding of the invention. However, it will be apparent that the invention may be practiced without these specific details. In order to avoid obscuring the present invention, some well-known chemistries and manufacturing processes are not disclosed in detail.

The drawings showing embodiments of the system are semi-diagrammatic and not to scale and, particularly, some of the dimensions are for the clarity of presentation and are shown exaggerated in the drawing FIGs. Similarly, although the views in the drawings for ease of description generally show similar orientations, this depiction in the FIGs. is arbitrary for the most part. Generally, the invention can be operated in any orientation.

Where multiple embodiments are disclosed and described having some features in common, for clarity and ease of illustration, description, and comprehension thereof, the same numbers are used in all the drawing FIGs. to relate to the same elements. The embodiments have been numbered first embodiment, second embodiment, etc. as a matter of descriptive convenience and are not intended to have any other significance or provide limitations for the present invention.

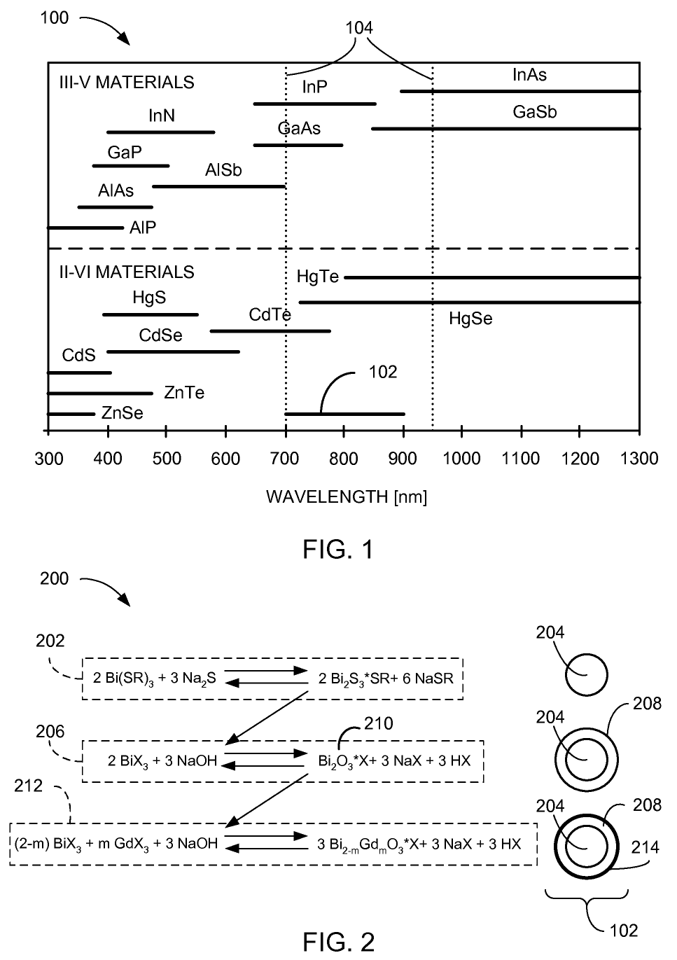

Referring now to FIG. 1, therein is shown a plot of luminescence emission wavelength 100 for a luminescent nanoparticle probe system 102 in one embodiment of the present invention compared to the emission of nanoparticles of different composition. The luminescent nanoparticle probe system 102 of FIG. 1 is a nanoparticle that includes bismuth sulfide (Bi₂S₃). Dotted lines 104 show the bounds of the optimal imaging range for in vivo applications.

As shown in FIG. 1, luminescent nanoparticle emissions span across a broad spectral range from ultra-violet (UV) to the infrared (IR). All visible and infrared colors shown can be excited with a single excitation wavelength. Only a limited number of materials can form near infra-red (NIR) nanoparticles with optimal wavelengths for in vivo detection, primarily indium phosphide (InP), gallium antimonide (GaSb), gallium arsenide (GaAs), mercury telluride (HgTe), mercury selenide (HgSe), cadmium telluride (CdTe) and the luminescent nanoparticle probe system 102 of the present invention. These compounds tend to oxidize readily in air and in aqueous environments, and therefore must be protected by an inert shell or coating. These coatings must be compatible with in biological environments.

Bismuth sulfide (Bi₂S₃) and other bismuth compounds are attractive materials for bioimaging. The optical band gap of bismuth sulfide is positioned in an ideal spectral range, as quantum confinement should shift the emission into the 700-850 nm range—the perfect near-infra-red range for deep in vivo imaging in tissues. This material is characterized by high stability and very low solubility in either aqueous or organic media. Furthermore, it stands out as a potentially non-toxic compound. Bismuth compounds have been used in pharmaceutical formulations for more than a century, to treat maladies such as diarrhea, syphilis, or peptic ulcers, as well as in cosmetics.

Synthesis of nanosized bismuth sulfide has been investigated mostly for potential applications in electronic and optoelectronic devices, with most reported syntheses focusing on nanorod, nanowire, and nanotube morphologies; however, there are no reports of the synthesis of bismuth sulfide nanoparticles that are consistent in mass, shape, and mass distribution (referred to as “monodisperse” bismuth sulfide nanoparticles).

Referring now to FIG. 2, therein is shown a synthesis scheme 200 for manufacturing the luminescent nanoparticle probe system 102 in one embodiment of the present invention. A first reaction 202 in the synthesis scheme 200 produces a bismuth sulfide core 204. A second reaction 206 coats the bismuth sulfide core 204 with a shell 208 preferably using an X-ray contrast material 210 such as bismuth oxide (Bi₂O₃). A third reaction introduces a shell dopant 214 on the surface of the shell 208, which is preferably a contrast material for magnetic resonance imaging such as galodinium (Gd³⁺).

In the first reaction 202, bismuth tri-mercaptoundecanoate, Bi(SR)₃ intermediate is prepared from bismuth citrate (2.5 mmol) and mercaptoundecanoic acid (7.5 mmol) by stirring in basic aqueous solution (600 mL). A number of bismuth salts can be used for the reaction including a number of anions that are generally regarded as safe (GRAS) such as citrate salts, cysteine salts, and acetate salts. Other marcaptoalkylcarboxilic acids may also be used. Dropwise addition of sodium sulfide (1.25 mmol in 100 mL) over 1.5 hours with vigorous overhead stirring results in a yellow to dark brown color change without a corresponding change in turbidity, signaling the formation of the bismuth sulfide core 204 rather than bulk bismuthinite. After stirring for 3 hours, the particles are purified by repeated ultrafiltration (30 kDa membrane) to yield a homogeneous solution of containing the bismuth sulfide core 204.

In the second reaction 206, the bismuth sulfide core 204 (10-100 mg of bismuth sulfide core nanoparticles) is added to an overhead stirred reactor at room temperature in 500 mL of water, and mixed with a solution of bismuth citrate (or other salt) at neutral pH. The molar ratio of bismuth citrate:core particles is determined based on the targeted shell thickness. Under vigorous stirring, the pH is slowly adjusted by addition of sodium hydroxide (NaOH) with a syringe pump until the pH reaches a level where precipitation of bismuth hydroxide/bismuth oxyhydroxide is known to occur at the concentrations used (established with previous range-finding experiments). Slow addition of further base at this point results in preferential shell growth on the existing core-particle surface.

Bismuth (Bi³⁺) is similar in size (1.03 Å) to galodinium (Gd³⁺) (0.938 Å), making it possible to dope the shell 208 with the shell dopant 214, which is preferably galodinum for enhanced MRI contrast. As part of the third reaction 212, the bismuth sulfide core 204 coated with the shell 208 (10-100 mg) is added to an overhead stirred reactor at room temperature in 500 mL of water, and mixed with a solution of bismuth citrate (or other salt) and galodinium citrate (or other salt) at neutral pH.

The stoichiometry of bismuth- and galodinium-salts can be altered as needed. Under vigorous stirring, the pH is slowly adjusted by addition of sodium hydroxide as done in the formation of the shell 208. The resulting luminescent nanoparticle probe system 102 is purified as before. In the luminescent nanoparticle probe system 102, the shell dopant 214 is preferably present at the exposed surface of the shell 208, where it can participate in water relaxation.

It has been discovered that the luminescent nanoparticle probe system 102 shown in FIG. 2 provides multimodality detection including luminescence X-ray contrast, and Magnetic Resonance Imaging contrast. Thus the luminescent nanoparticle probe system 102 of the present invention may be used in conjunction with optical imaging, X-ray imaging (including computed tomography), and magnetic resonance equipment or multi-modality equipment incorporating two or more of these means of detection.

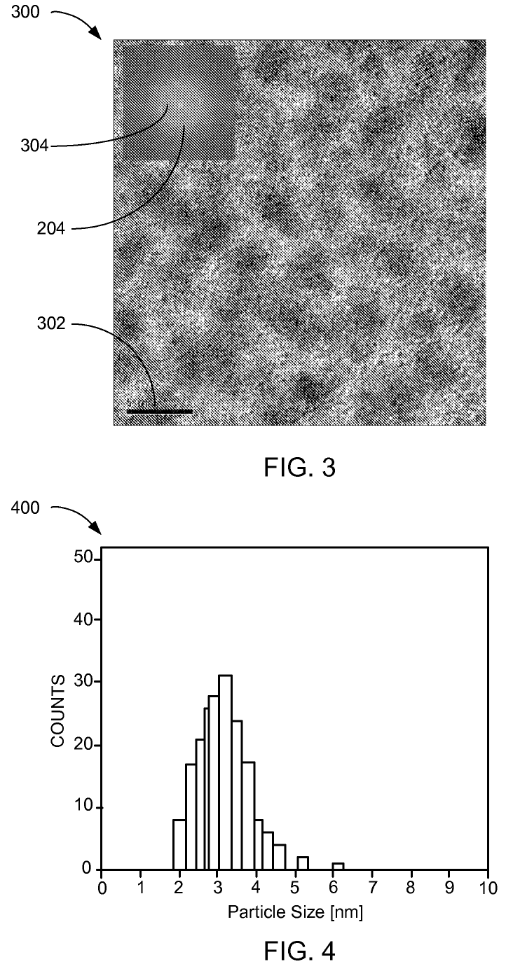

Referring now to FIG. 3, therein is shown a high-resolution Transmission Electron Microscope (TEM) image 300 showing the bismuth sulfide core 204 in the luminescent nanoparticle probe system 102 of the present invention. A scale bar 302 shows a relative scale of 5 nm. An insert 304 shows a Fourier Transformation of a corresponding part of the high-resolution Transmission Electron Microscope (TEM) image 300 with the bismuth sulfide core 204. The high-resolution Transmission Electron Microscope (TEM) image 300 exhibits clear lattice fringes revealing the high crystallinity of the bismuth sulfide core 204.

Referring now to FIG. 4, therein is a size distribution plot 400 of the bismuth sulfide core 204. The synthesis scheme 200 of FIG. 2 produces a nearly spherical and nearly size-uniform-bismuth sulfide core 204 in the size range of 3-5 nm. Particle size may be controlled by adjusting the relative amounts of reactants in the first reaction 202 of the synthesis scheme 200.

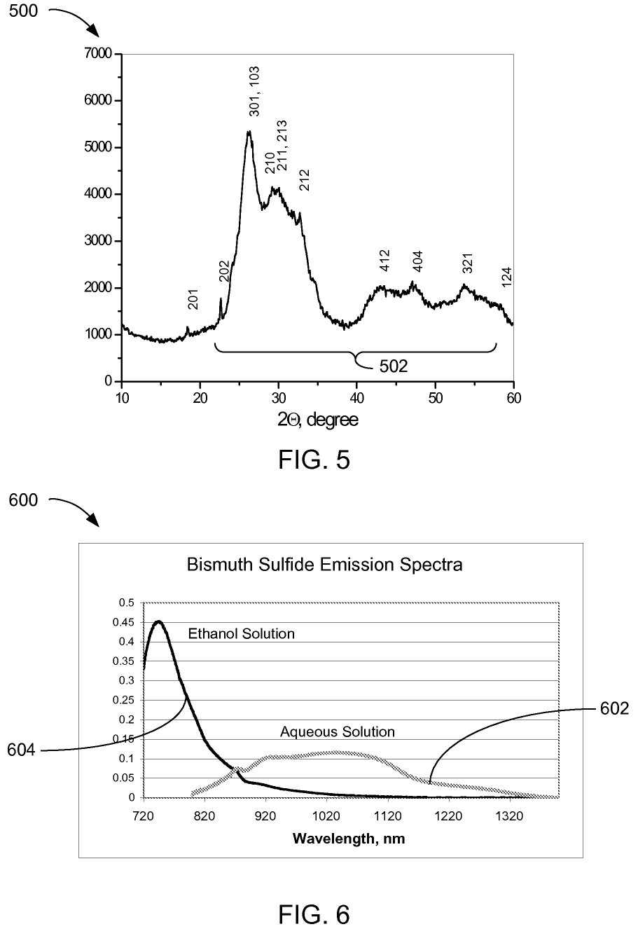

Referring now to FIG. 5, therein is shown X-ray diffraction data 500 for the bismuth sulfide core 204 of the luminescent nanoparticle probe system 102 of the present invention. Diffraction peaks 502 in the X-ray diffraction data 500 indicate an orthorhombic bismuth sulfide structure. The broadening of the diffraction peaks 502 is ascribed to the small particle size effect, since the width of the diffraction peak exhibits inverse dependence to crystallite size.

Referring now to FIG. 6, therein is shown fluorescence emission spectra 600 of the bismuth sulfide core 204 in the luminescent nanoparticle probe system 102 of the present invention. Aqueous emission 602 of the bismuth sulfide core 204 displays a broad peak at 1050 nm and is relatively dim presumably due to “deep-trap” emission. In contrast, ethanol emission 604 has a narrow, bright-band edge emission with a peak at approximately 750 nm, due to surface exchange and ethanolic dispersion.

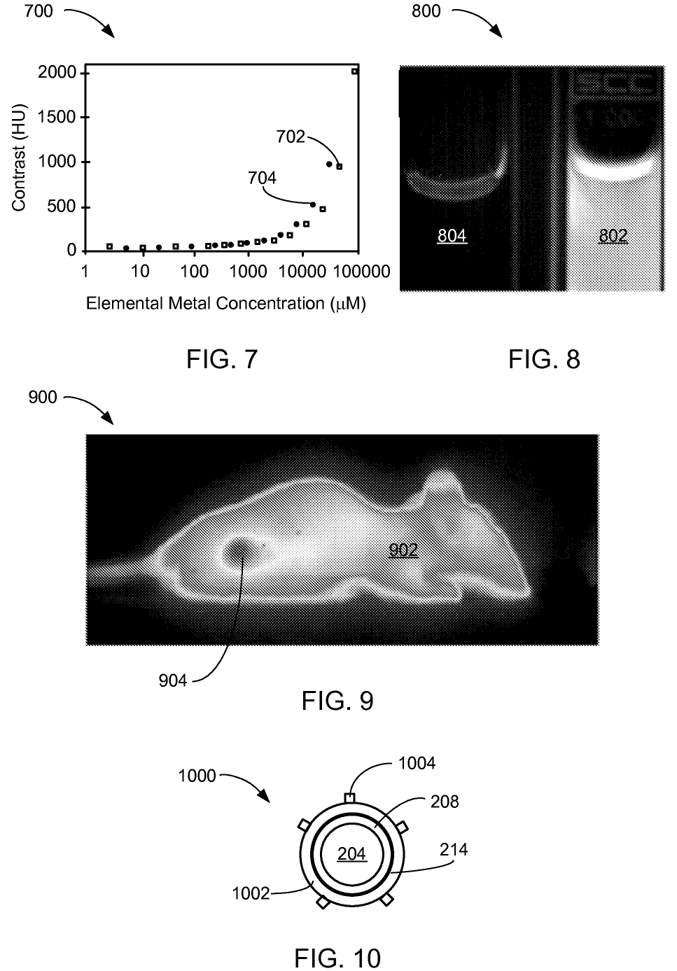

Referring now to FIG. 7, therein is shown X-ray (CT) contrast data 700 for the bismuth sulfide core 204 compared to that of a cadmium telluride nanoparticle. The X-ray (CT) contrast data 700 includes bismuth sulfide contrast 702 and cadmium telluride nanoparticle contrast 704. The bismuth sulfide contrast 702 provided by the bismuth sulfide core is comparable to the cadmium telluride nanoparticle contrast 704.

Referring now to FIG. 8, therein is shown an image 800 showing fluorescence of the bismuth sulfide core 204 in the luminescent nanoparticle probe system 102. The bismuth sulfide core fluorescence 802 is easily detectable compared to a water-filled cuvette signal 804.

Referring now to FIG. 9, therein is shown an image 900 of a nude mouse 902 that has been administered intradermally a saline solution containing the bismuth sulfide core 204 (i.e., a suspension of these nanoparticles). A fluorescence signal 904 from the bismuth sulfide core 204 is clearly seen at the injection site for the saline solution containing the bismuth sulfide core 204.

Referring now to FIG. 10, therein is shown a schematic diagram of a luminescent nanoparticle probe system 1000 in one embodiment of the present invention. The luminescent nanoparticle probe system 1000 includes the bismuth sulfide core 204, the shell 208, and the shell dopant 214 on the surface of the shell 208. In addition, in this embodiment of the invention the luminescent nanoparticle probe system 1000 includes a stabilization layer 1002 with a functional molecule 1004 on the surface of the stabilization layer 1002. The stabilization layer 1002 may be implemented using mercaptoalkylacetic acid ligands or modified mercaptoalkylacetic acid, in both cases with alkyl group number ranging from 1 to 16.

The stabilization layer 1002 provides passivation, compatibility with external biological environments, and long-term stability for luminescent nanoparticle probe system 1000. The functional molecule 1004 is an attachment point for conjugations or complexing to elements external to the luminescent nanoparticle probe system 1000.

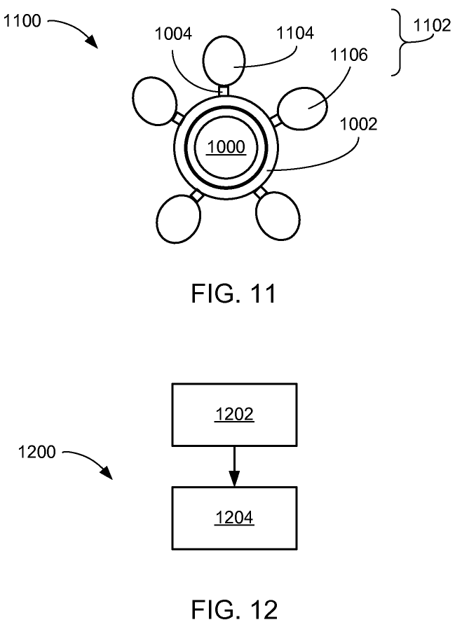

Referring now to FIG. 11, therein is shown a luminescent nanoparticle probe system 1100 incorporating the luminescent nanoparticle probe system 1000 of FIG. 10 and an external molecule 1102. In this embodiment of the present invention, the external molecule 1102 may be a biomolecule 1104 or an energy donor 1106.

The biomolecule 1104 is any organic or biological molecule without limitation including peptides, proteins such as enzymes, fusion proteins, and antibodies, poly-nucleotides such as DNA and RNA, biotin, and sugars. The energy donor 1106 is any nanoparticle, biomolecule, dye, polymer, or chemical entity capable of acting as an energy donor in an energy transfer process between the energy donor 1106 and the luminescent nanoparticle probe system 1000. Examples of energy transfer processes include Fluorescence Resonance Energy Transfer (FRET, where the energy donor is a fluorescent entity), Bioluminescence Resonance Energy Transfer (BRET, with a bioluminescent protein acting as the energy donor), or Chemiluminescence Resonance Energy Transfer (CRET, with a chemiluminescent entity acting as the energy donor).

Referring now to FIG. 12, therein is shown a flow chart of a method 1200 of manufacture of a luminescent nanoparticle probe system 102 in a further embodiment of the present invention. The method 1200 includes: forming a bismuth sulfide core in a block 1202; and coating the bismuth sulfide core with a shell in a block 1204.

The resulting method, process, apparatus, device, product, and/or system is straightforward, cost-effective, uncomplicated, highly versatile, accurate, sensitive, and effective, and can be implemented by adapting known components for ready, efficient, and economical manufacturing, application, and utilization.

Another important aspect of the present invention is that it valuably supports and services the historical trend of reducing costs, simplifying systems, and increasing performance.

These and other valuable aspects of the present invention consequently further the state of the technology to at least the next level.

While the invention has been described in conjunction with a specific best mode, it is to be understood that many alternatives, modifications, and variations will be apparent to those skilled in the art in light of the aforegoing description. Accordingly, it is intended to embrace all such alternatives, modifications, and variations that fall within the scope of the included claims. All matters hithertofore set forth herein or shown in the accompanying drawings are to be interpreted in an illustrative and non-limiting sense.

What is claimed is:

1. A method of manufacture of a luminescent nanoparticle probe system comprising:
forming a bismuth sulfide core; and
coating the bismuth sulfide core with a shell.

2. The method as claimed in claim 1 wherein coating the bismuth sulfide core with the shell is done using an X-ray contrast material.

3. The method as claimed in claim 1 further comprising doping the shell with a shell dopant that provides contrast for magnetic resonance imaging.

4. The method as claimed in claim 1 further comprising coating the shell with a stabilization layer.

5. The method as claimed in claim 1 further comprising coupling a biomolecule to the stabilization layer through a functional molecule in the stabilization layer.

6. A method of manufacture of a luminescent nanoparticle probe system comprising:
forming a bismuth sulfide core;
coating the bismuth sulfide core with a shell; and
coating the shell with a stabilization layer.

7. The method as claimed in claim 6 wherein coating the bismuth sulfide core with a shell is done using an X-ray contrast material.

8. The method as claimed in claim 6 further comprising incorporating a shell dopant within the shell wherein the shell dopant provides contrast for magnetic resonance imaging.

9. The method as claimed in claim 6 further comprising coupling a biomolecule to the stabilization layer through a functional molecule in the stabilization layer.

10. The method as claimed in claim 6 further comprising coupling an energy donor to the stabilization layer through a functional molecule in the stabilization layer.

11. A luminescent nanoparticle probe system comprising:
a bismuth sulfide core; and
a shell coating the bismuth sulfide core.

12. The system as claimed in claim 11 wherein the shell includes an X-ray contrast material.

13. The system as claimed in claim 11 further comprising a shell dopant that provides contrast for nuclear magnetic resonance imaging.

14. The system as claimed in claim 11 further comprising a stabilization layer coating the shell.

15. The system as claimed in claim 11 further comprising a biomolecule coupled to the stabilization layer through a functional molecule in the stabilization layer.

16. The system of claim 11 further comprising a stabilization layer coating the shell.

17. The system as claimed in claim 16 wherein the shell includes an X-ray contrast material.

18. The system as claimed in claim 16 further comprising a shell dopant incorporated within the shell that provides contrast for nuclear magnetic resonance imaging

19. The system as claimed in claim 16 further comprising coupling a biomolecule to the stabilization layer through a functional molecule in the stabilization layer.

20. The system as claimed in claim 16 further comprising an energy donor coupled to the stabilization layer through a functional molecule in the stabilization layer.
